(19) United States
(12) Patent Application Publication
(10) Pub. No.: US 2022/0008202 A1
(43) Pub. Date: Jan. 13, 2022

(54) TUBULAR INDWELLING DEVICE

(71) Applicant: KAWASUMI LABORATORIES, INC., Saiki-shi, Oita (JP)

(72) Inventor: Noriaki Shirahama, Bungo-ono-shi, Oita (JP)

(21) Appl. No.: 17/291,561

(22) PCT Filed: Nov. 18, 2019
(86) PCT No.: PCT/JP2019/045043

§ 371 Date: May 5, 2021

(30) Foreign Application Priority Data
Nov. 19, 2018 (JP) 2018-216663

(51) Int. Cl. A61F 2/24; A61F 2/90

(52) U.S. Cl. CPC A61F 2/2476; A61F 2002/041; A61F 2/90

(57) ABSTRACT

A tubular indwelling device 1 is placed inside a living body lumen and defines a tubular flow path, the tubular indwelling device comprising: a skeleton part 10; a membrane part 20; and a shape retention part 30. The skeleton part 10 includes a body part 11 which can expand/contract along the radial direction of the tubular indwelling device 1. The membrane part 20 is provided along the body part 11, and includes a protruding part 22 that protrudes from a tube end part 11a so as to create a tapered shape in which a flow path cross-section area on a distal end side spaced apart from the tube end part 11a is smaller than a flow path cross-section area on a base end side which is the tube end part 11a side of the body part 11. The shape retention part 30 retains the shape of a flow outlet 23 of the protruding part 22.

### CROSS-REFERENCE TO RELATED APPLICATIONS

This application is a national stage filing under 35 U.S.C. 371 of PCT/JP2019/045043, filed Nov. 18, 2019, which International Application was published by the International Bureau in English on May 28, 2020, as WO 2020/105579, and application claims priority from Japanese Application No. 2018-216663, filed on Nov. 19, 2018, which applications are hereby incorporated in their entireties by reference in this application.

### BACKGROUND OF THE INVENTION

### Field of the Invention

The present invention relates to a tubular indwelling device which is placed inside a living body lumen.

### Description of the Related Art

Conventionally, tubular indwelling devices which are placed inside a living body lumen such as a blood vessel or a bile duct are known. This type of tubular indwelling device generally has a tubular shape, and comprises a skeleton part which can expand and contract in a radial direction, and a membrane part which is provided along the skeleton part.

For example, in one tubular indwelling device used in the treatment of bile duct stenosis or obstruction, the membrane part has a body part provided along the skeleton part, and a cylindrical protruding part that protrudes in a cylindrical shape from one end of the body part (for example, see Japanese Unexamined Patent Application Publication No. H7-275369). This conventional tubular indwelling device is used by placing the body part of the membrane part inside the bile duct such that the cylindrical protruding part of the membrane part extends into the duodenum.

In order to properly suppress the discharge of bile from the bile duct into the duodenum, and the backflow of foreign substances from the duodenum into the bile duct, it is desirable to ensure that the opening of the cylindrical protruding part is closed when bile is not being released from the gall bladder. However, in the conventional tubular indwelling device for the bile duct described above, the cylindrical protruding part merely extends into the duodenum, and the open/closed state of the opening of the cylindrical protruding part is not actively managed. It is desirable for tubular indwelling devices provided with the check valve-like function described above (hereinafter referred to as “valve function”), which are not limited to tubular indwelling devices for the bile duct, to properly achieve the valve function.

### SUMMARY OF THE INVENTION

A tubular indwelling device according to the present invention is placed inside a living body lumen and defines a tubular flow path, the tubular indwelling device comprising: a skeleton part which includes a body part having a tubular structure that can expand and contract along a radial direction of the tubular indwelling device; a membrane part which is provided along the body part, and includes a protruding part that protrudes from a tube end part so as to create a tapered shape in which a flow path cross-section area on a distal end side which is spaced apart from the tube end part is smaller than a flow path cross-section area on a base end side which is at the tube end part side of the body part; and a shape retention part that retains a shape of a flow outlet of the protruding part.

The present invention has been briefly described above. Further, the details of the present invention will be further clarified by reading through a mode for carrying out the invention described below (hereinafter referred to as “embodiment”) with reference to the accompanying drawings.

### BRIEF DESCRIPTION OF THE DRAWINGS

FIG. 1A is a perspective view of an end part of a tubular indwelling device according to an embodiment of the present invention; FIG. 1B is an enlarged view of the surroundings of a flow outlet of the tubular indwelling device when the flow outlet is in a closed state; and FIG. 1C is an enlarged view of the surroundings of the flow outlet of the tubular indwelling device when the flow outlet is in an open state.

FIG. 2 is a top view of the end part of the tubular indwelling device shown in FIG. 1.

FIG. 3 is a diagram for describing the relationship between a flow path cross-section of a body part of a membrane part, and a flow path cross-section of a flow outlet of a protruding part of the membrane part.

FIG. 4 is a diagram of an end part of a tubular indwelling device according to a modification of the embodiment of the present invention, and corresponds to FIG. 1B.

FIG. 5 is a perspective view of an end part of a tubular indwelling device according to another modification of the embodiment of the present invention.

### DESCRIPTION OF THE EMBODIMENT

### Embodiment

Hereinafter, a tubular indwelling device 1 according to an embodiment of the present invention will be described with reference to the drawings.

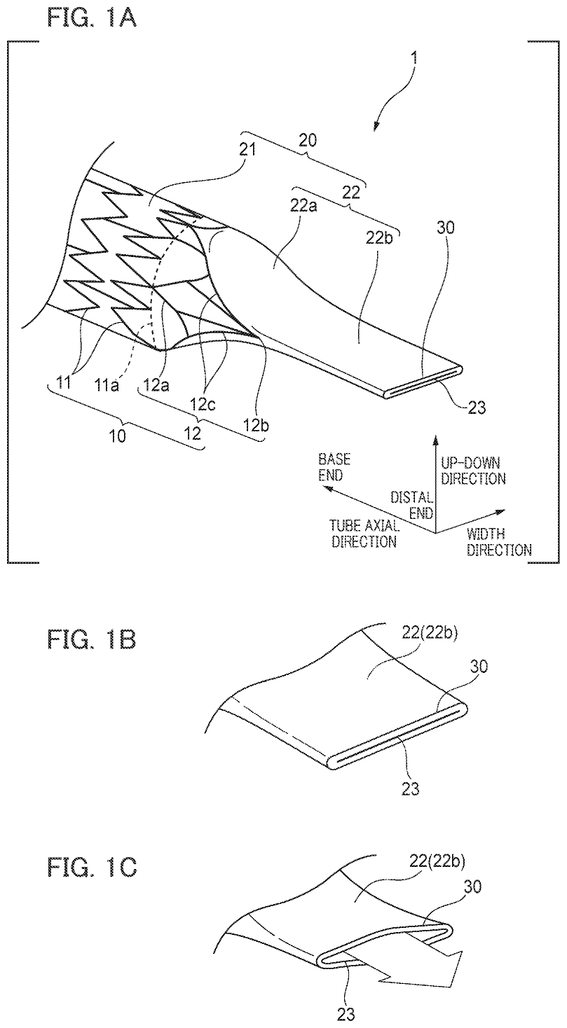

For convenience of the description, as shown in FIG. 1A, the longitudinal direction of the tubular indwelling device 1 is referred to as the “tube axial direction”, one of the directions orthogonal to the “tube axial direction” is referred to as the “width direction”, and the direction orthogonal to the “tube axial direction” and the “width direction” is referred to as the “up-down direction”. Furthermore, after the tubular indwelling device 1 has been placed, one end side in the “tube axial direction” (gall bladder side) is referred to as the “base end side”, and the other end side (duodenum side) is referred to as the “distal end side”.

The tubular indwelling device 1 is used to treat lesions such as an obstruction or stenosis of the bile duct by outwardly expanding the lesion in the radial direction. The tubular indwelling device 1 is typically used by being placed inside the bile duct so that the base end side and the distal end side respectively face the gall bladder side and the duodenum side. The tubular indwelling device 1 is also commonly referred to as a bile duct stent. Hereinafter, a state in which the tubular indwelling device 1 has been placed in a lesion of the bile duct is referred to as “at the time of bile duct placement”.

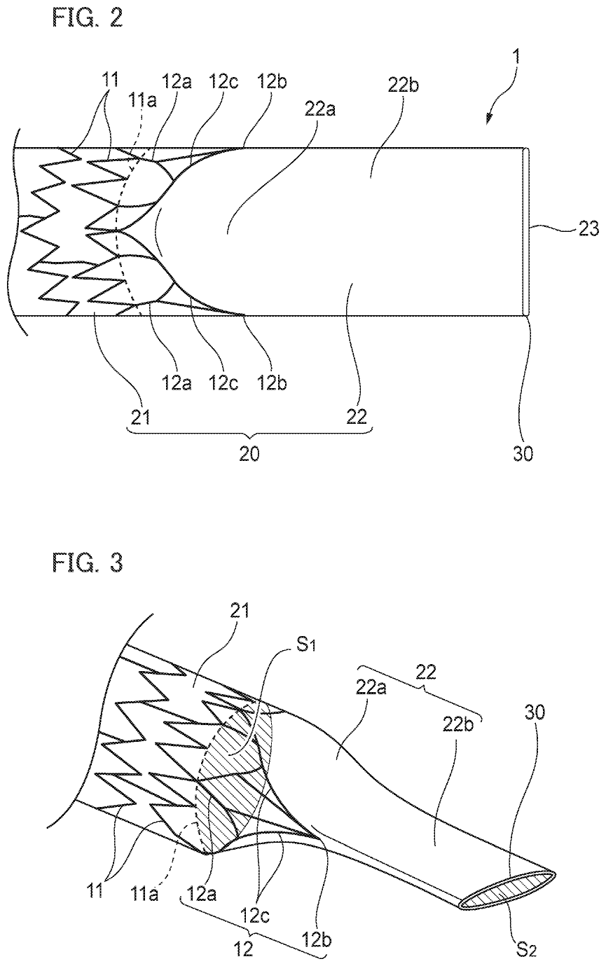

As shown in FIG. 1A to FIG. 1C, and FIG. 2, the tubular indwelling device 1 includes a skeleton part 10 and a membrane part 20. Hereinafter, the configuration of these will be described in turn.

First, the skeleton part 10 will be described.

The skeleton part 10 is configured to be self-expandable, and in the present example, includes a body part 11 having a tubular structure for defining a flow path for conduction of a fluid such as bile, and a pair of extension parts 12 provided so as to extend from a tube end part 11a of the body part 11.

The body part 11 has a plurality of zigzag annular parts arranged side-by-side in the tube axial direction which are configured by metal wires extending in an annular shape in the circumferential direction while reciprocating in a zigzag shape in the tube axial direction. In the body part 11, adjacent zigzag annular parts are joined to each other in the tube axial direction by metal wires at a plurality of locations in the circumferential direction. The body part 11 has a cylindrical shape as a whole.

The tube end part 11a, for example, is the end part of the body part 11 on the duodenum side (distal end side). As indicated by the broken line in the figure, the tube end part 11a also corresponds to a boundary that separates the body part 11 and the pair of extension parts 12 and 12.

The pair of extension parts 12 and 12 are made of metal wires, and are configured so as to extend from both width direction sides of the body part 11 toward the distal end side in the tube axial direction. That is to say, the pair of extension parts 12 and 12 are arranged so as to face each other across the tube axis of the tubular indwelling device 1. In the present example, the pair of extension parts 12 and 12 are configured such that the width in the up-down direction gradually becomes smaller moving away from the body part 11. Each of the pair of extension parts 12 and 12 includes a joined part 12a which is connected to a predetermined location of the body part 11, an apex 12b which is located farthest on the distal end side in the tube axial direction, and a V-shaped section 12c which extends diagonally upward and diagonally downward from the apex 12b toward the base end side in the tube axial direction.

As described below, the pair of extension parts 12 and 12 function as supporting members that support a protruding part 22 of a membrane part 20. A force that opens the protruding part 22 in the width direction may be exerted on the protruding part 22 as a result of the pair of extension parts 12 and 12 expanding in a direction away from each other. Alternatively, the pair of extension parts 12 and 12 may not exert such a force on the protruding part 22.

The skeleton part 10 is configured to be expandable from a contracted state, which is inwardly contracted in the radial direction, to an expanded state, which is outwardly expanded in the radial direction. When the skeleton part 10 is in the expanded state, the tubular indwelling device 1 defines a cylindrical flow path inside the tubular indwelling device 1. For example, the skeleton part 10 is configured such that it expands in the tube axial direction while inwardly contracting in the radial direction as a result of being pulled in the tube axial direction, and contracts in the tube axial direction while outwardly expanding in the radial direction as a result of being released from the contracted state. As a result of the skeleton part 10 being configured in this manner, at the time of bile duct placement, the outer circumferential surface of the skeleton part 10, and in particular, the outer circumferential surface of the body part 11 outwardly presses the inner surface of the lesion of the bile duct in the radial direction, which enables the lesion of the bile duct to be outwardly pressed and expanded in the radial direction.

Examples of the material constituting the skeleton part 10 include known metals or metal alloys typified by stainless steel, Ni—Ti alloy (that is to say, nitinol), titanium alloy, and the like. Furthermore, part or all of the skeleton part 10 may be made of an alloy material having an X-ray contrast property so that the position of the skeleton part 10 can be confirmed from outside of the body. The skeleton part 10 may be made of a material other than a metallic material, such as a ceramic or a resin.

The skeleton part 10 can be produced, for example, by a method in which all sections except for the section corresponding skeleton part 10 are cut away from a raw material pipe made of Ni—Ti alloy using a laser or the like. Furthermore, the skeleton part 10 can be produced, for example, by weaving a thin wire made of Ni—Ti alloy into a shape corresponding to the skeleton part 10. When Ni—Ti alloy is used as the material constituting the skeleton part 10, the skeleton part 10 is adjusted to the shape of the expanded state shown in FIG. 1A, and then the shape can be memorized by the skeleton part 10 by performing a predetermined heat treatment. As a result, an expandable skeleton part 10 which is capable of changing shape from the contracted state to the expanded state can be formed.

The material of the metal wire constituting the skeleton part 10, the type of wire (for example, a wire such as a circular wire or a square wire formed by laser cutting), the wire diameter (cross-section area), the number of zigzag round-trips in the circumferential direction, the shape of the zigzag shape, and the spacing between wires in the tube axial direction (skeleton amount per unit length), and the like can be appropriately selected according to the living body lumen in which the device is to be placed.

Next, the membrane part 20 will be described.

As shown in FIG. 1 and FIG. 2, the membrane part 20 has a configuration in which a cylindrical part 21, which is provided along the body part 11 of the skeleton part 10, and a protruding part 22, which protrudes from the end part of the cylindrical part 21, are integrally connected. Examples of the material constituting the membrane part 20 include silicone resins, fluorine-based resins such as PTFE (polytetrafluoroethylene), and polyethylene resins such as polyethylene terephthalate.

The cylindrical part 21 is a film body provided along the body part 11. When the body part 11 is in the expanded state at the time of bile duct placement, the cylindrical part 21 defines a flow path that guides bile toward the protruding part 22. The cylindrical part 21 may be arranged on the outer circumferential surface and the inner circumferential surface of the body part 11 so as to sandwich the body part 11, arranged on only the outer circumferential surface of the body part 11, or arranged on only the inner circumferential surface of the body part 11. The cylindrical part 21, for example, can be fixed to the body part 11 by using a known method such as stitching or dipping.

The protruding part 22 is a film body of the membrane part 20 that continuously protrudes from the distal side end part of the cylindrical part 21 toward the distal end side in the tube axial direction. At the time of bile duct placement, the protruding part 22 is a section that discharges bile toward the duodenum. The protruding part 22 as a whole has a tapered shape in which the flow path cross-section area on the distal end side which is spaced apart from the cylindrical part 21 is smaller than the flow path cross-section area on the base end side connected to the cylindrical part 21. More specifically, in the present example, the protruding part 22 includes a first section 22a, in which the flow path cross-section area gradually becomes smaller along the pair of extension parts 12 and 12 of the skeleton part 10 from the base end side toward the distal end, and a second section 22b, which extends from the first section 22a toward the distal end side and has a substantially constant flow path cross-section area. In the second section 22b, the film body constituting the protruding part 22 is in a substantially adhered state in the up-down direction. The skeleton part 10 is not provided in the second section 22b. The membrane part 20 having such a shape can be formed by using a known method such as dipping.

The opening in the end part of the protruding part 22 in the tube axial direction distal side functions as a flow outlet 23, which causes fluid such as bile that has flowed into the protruding part 22 from the cylindrical part 21 to be discharged into the duodenum.

As shown in FIG. 1B, the flow outlet 23 of the protruding part 22 linearly extends in the width direction and maintains a closed state when a fluid is not flowing inside the tubular indwelling device 1. On the other hand, when a fluid is flowing inside the tubular indwelling device 1, as shown in FIG. 1C, the flow outlet 23 vertically opens due to the pressure of the fluid itself. As a result, at the time of bile duct placement, the protruding part 22 achieves a check valve-like function, which suppresses the discharge of bile from the bile duct into the duodenum, and the backflow of foreign substances from the duodenum into the bile duct.

When the flow outlet 23 of the protruding part 22 “closes”, this indicates a change in shape of the protruding part 22 such that the aperture area of the flow outlet 23 decreases. Specifically, the shape of the protruding part 22 may change to an extent that causes the aperture area of the flow outlet 23 to become substantially zero. Alternatively, the shape of the protruding part 22 may change such that the aperture area of the flow outlet 23 becomes a predetermined aperture area which is smaller than the aperture area of the state shown in FIG. 1C, but is larger than zero.

A shape retention part 30 for retaining the shape of the flow outlet 23 is provided on the end part of the protruding part 22 in the tube axial direction distal side.

The shape retention part 30 is a ring-shaped member which is flat in the width direction, and is for retaining the shape of the flow outlet 23 in the closed state so that it linearly extends in the width direction. The shape retention part 30 has a suitable elasticity and rigidity so that the flow outlet 23 can be opened and closed. Furthermore, when a fluid such as bile flows from the cylindrical part 21 into the protruding part 22, the shape retention part 30 is configured such that it is capable of opening due to the pressure of the fluid itself, and properly closing the flow outlet 23 when such a fluid is not flowing in. The shape retention part 30 may be made of a resin such as silicone, or may be made of a metal. The shape retention part 30 is secured to the end part of the protruding part 22 in the tube axial direction distal side by a known method such as stitching or adhesion to the protruding part 22.

Furthermore, the shape retention part 30 regulates the flow outlet 23 such that a flow path cross-section area S2, which is formed when the flow outlet 23 opens as a result of a fluid such as bile flowing in from the cylindrical part 21 into the protruding part 22, falls within a predetermined range relative to a flow path cross-section area S1 of the cylindrical part 21 in the expanded state of the skeleton part 10. That is to say, the shape retention part 30 sets the aperture area (flow path cross-section area S2) of the flow outlet 23 in consideration of the flow rate of the fluid flowing through the cylindrical part 21. As a result, the open/closed state of the flow outlet 23 can be more properly managed.

In this manner, the tubular indwelling device 1 according to the present embodiment is placed in a living body lumen and defines a tubular flow path, the device comprising: a skeleton part 10 which includes a body part 11 having a tubular structure that can expand and contract along a radial direction of the tubular indwelling device 1, a membrane part 20 which is provided along the body part 11, and includes a protruding part 22 that protrudes from a tube end part 11a so as to create a tapered shape in which a flow path cross-section area S2 on a distal end side which is spaced apart from the tube end part 11a is smaller than a flow path cross-section area S1 on a base end side, which is the tube end part 11a side of the body part 11; and a shape retention part 30 that retains a shape of a flow outlet 23 of the protruding part 22.

Therefore, as a result of the shape retention part 30 retaining the shape of the flow outlet 23 of the protruding part 22, which protrudes from the tube end part 11a such that it has the tapered shape of the membrane part 20, the open/closed state of the flow outlet 23 can be properly managed irrespective of the shape of the protruding part 22. As a result, a valve function having a backflow suppressing effect can be more properly exhibited.

In particular, for example, when the membrane part 20 has a sufficient flexibility, the inner surfaces of the protruding part 22, which has a tapered shape, can sometimes naturally adhere to each other due to the surface tension of the bile and the like which is present inside the protruding part 22. In this case, the flow outlet 23 is blocked by the protruding part 22 itself. In this state, it is generally more difficult for backflow to occur through the flow outlet 23 compared to a case where the adhesion mentioned above does not occur. The longer the second section 22b (a so-called blow-through part) on the distal end side of the protruding part 22 is in the tube axial direction, the longer the region in which the inner surfaces of the protruding part 22 can adhere to each other, and therefore, the backflow suppression effect due to the protruding part 22 itself is considered to increase. In contrast, when the length of the second section 22b on the distal end side of the protruding part 22 is made shorter due to requirements such as reduction of the size of the tubular indwelling device 1, the backflow suppression effect due to the protruding part 22 may decrease. Here, the tubular indwelling device 1 is configured such that the shape retention part 30 retains the shape of the flow outlet 23. Therefore, a valve function having a backflow suppressing effect can be more properly exhibited irrespective of the length of the protruding part 22, and in particular, the length of the second section 22b on the distal end side.

Further, by designing the shape and the like of the shape retention part 30 such that the flow outlet 23 can be retained in the closed state when a fluid such as bile is not flowing inside the tubular indwelling device 1, and enabling the flow outlet 23 to be opened by the pressure of the fluid itself when a fluid is flowing inside the tubular indwelling device 1, the valve function described above can be more properly exhibited.

In addition, by providing the shape retention part 30 on the distal end side where the flow outlet 23 of the protruding part 22 of the membrane part 20 is provided, the shape retention part 30 can be arranged near the flow outlet 23. As a result, the open/closed state of the flow outlet 23 can be more properly managed, and a valve function having a backflow suppressing effect can be more properly exhibited.

Also, because the skeleton part 10 includes the pair of extension parts 12 and 12 that support the protruding part 22, and the pair of extension parts 12 and 12 are arranged so as to face each other across the tube axis of the tubular indwelling device 1, the tapered shape of the protruding part 22 can be more properly maintained.

### Other Forms

The present invention is not limited to the embodiments described above, and various modifications can be adopted within the scope of the present invention. For example, the present invention is not limited to the embodiment described above, and appropriate modifications, improvements, and the like are possible. In addition, the materials, shapes, dimensions, numbers, arrangement positions, and the like of each of the components in the embodiment described above are arbitrary and are not limited as long as the present invention can be achieved.

In the embodiment described above, a shape retention part 30 provided on the distal side end part of the protruding part 22 of the membrane part 20 has been exemplified. However, the embodiment described above is an example and is not limited to this, and the position of the shape retention part 30 can be arbitrarily changed as appropriate. Furthermore, a shape retention part 30 having a flat ring shape in the width direction has been exemplified. However, the embodiment described above is an example and is not limited to this, and the shape of the shape retention part 30 can be arbitrarily changed as appropriate.

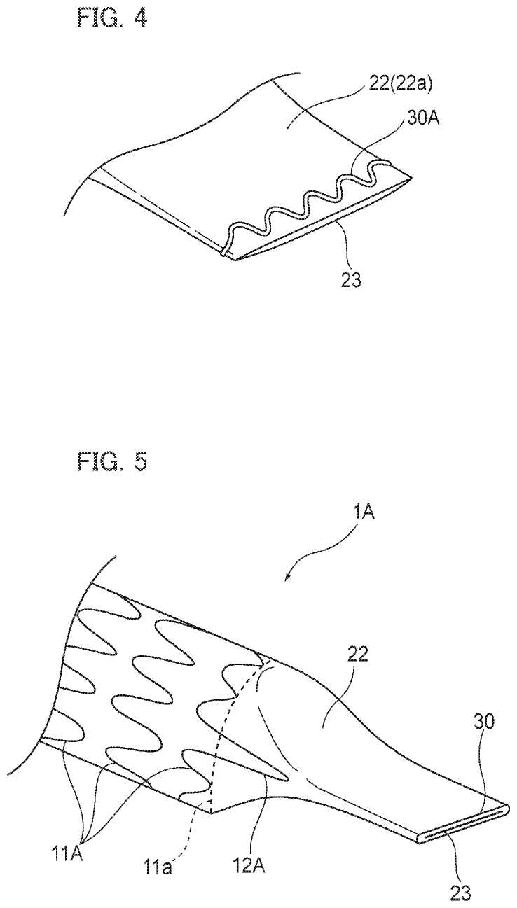

For example, as shown in FIG. 4, the shape retention part 30A may be provided at a position slightly closer to the base end side than the distal side end part of the protruding part 22 of the membrane part 20, and may be a ring-shaped member that extends in the width direction while reciprocating in a zigzag shape in the tube axial direction.

Moreover, the shape retention part 30 may be configured by a plurality of members rather than a single member.

Further, in the embodiment described above, the shape retention part 30 is not connected to the skeleton part 10. In contrast, the shape retention part 30 may be connected to the skeleton part 10 (for example, to the pair of apices 12b). Specifically, for example, the pair of apices 12b of the skeleton part 10 and both end parts of the shape retention part 30 in the width direction may be connected to each other.

In addition, in the embodiment described above, the protruding part 22 of the membrane part 20 has a first section 22a on the base end side, and a second section 22b on the distal end side. In contrast, the protruding part 22 may be configured to not include the second section 22b. Furthermore, in the embodiment described above, as shown in FIG. 2, the protruding part 22 has substantially the same width in the tube axial direction. In contrast, the protruding part 22 may have a shape in which the width on the distal end side is smaller than the width on the base end side. Moreover, in the embodiment described above, the flow outlet 23 of the protruding part 22 has a flat shape extending substantially linearly in the width direction. In contrast, for example, the flow outlet 23 may be configured so as to extend in a diagonal direction that intersects the tube axial direction.

Also, the structure of the body part 11 of the skeleton part 10 is not necessarily limited to a structure in which a plurality of zigzag annular portions is arranged side-by-side in the tube axial direction. For example, like the tubular indwelling device 1A shown in FIG. 5, the body part 11A may be configured such that a metal wire is spirally wound while reciprocating in a zigzag shape in the tube axial direction. In addition, in this case, the pair of extension parts 12A and 12A may be configured such that a portion of the metal wire which is wound in the manner above extends toward the flow outlet 23. Even in this case, a force that opens the protruding part 22 in the width direction may be exerted on the protruding part 22 as a result of the pair of extension parts 12A and 12A expanding in a direction away from each other. Alternatively, the pair of extension parts 12A and 12A may not exert such a force on the protruding part 22.

Further, in the embodiment described above, the tubular indwelling device 1 is used by being placed in the bile duct. However, the tubular indwelling device 1 may be used with respect to other living body lumens that require a valve function having a backflow suppressing effect to be more properly exhibited. Alternatively, the tubular indwelling device 1 may also be used with respect to other living body lumens in which such a valve function is not required.

What is claimed is:

1. A tubular indwelling device which is placed inside a living body lumen and defines a tubular flow path, the tubular indwelling device comprising:
a skeleton part which includes a body part having a tubular structure that can expand and contract along a radial direction of the tubular indwelling device;
a membrane part which is provided along the body part, and includes a protruding part that protrudes from a tube end part of the body part so as to create a tapered shape in which a flow path cross-section area on a distal end side which is spaced apart from the tube end part is smaller than a flow path cross-section area on a base end side which is at the tube end part side of the body part; and
a shape retention part that retains a shape of a flow outlet of the protruding part.

2. The tubular indwelling device according to claim 1, wherein
in the shape retention part, the flow outlet is retained in a closed state when a fluid is not flowing inside the tubular indwelling device, and the flow outlet is opened by a pressure of the fluid when the fluid is flowing inside the tubular indwelling device.

3. A tubular indwelling device according to claim 1, wherein
the shape retention part is provided on the distal end side where the flow outlet of the protruding part is provided.

4. A tubular indwelling device according to claim 1, wherein
the skeleton part includes a pair of extension parts that support the protruding part by being provided so as to extend from the tube end part of the body part, and
the pair of extension parts are arranged so as to face each other across a tube axis of the tubular indwelling device.
